(19) United States
(12) Patent Application Publication
(10) Pub. No.: US 2025/0000190 A1
(43) Pub. Date: Jan. 2, 2025

(54) Active Device Glove, and Method Enabling Use of Active Technology In Gloves

(71) Applicant: Richard D Ward, Savannah, GA (US)

(72) Inventor: Richard D Ward, Savannah, GA (US)

(21) Appl. No.: 18/882,941

(22) Filed: Sep. 12, 2024

Related U.S. Application Data
(63) Continuation-in-part of application No. 17/756,337, filed on May 23, 2022.

(51) Int. Cl. A41D 19/00; A61B 5/00

(52) U.S. Cl. CPC A41D 19/0027; A41D 19/0037; A61B 5/6806; A61B 5/681

(57) ABSTRACT

This invention relates to a glove, mitten, or sleeve that securely holds an Active Technology Device (ATD), such as a smartwatch or fitness tracker, during sports activities. The ATD is positioned in the webbing between the thumb and forefinger at an optimal viewing angle, ensuring continuous visibility and minimal interference with hand movements. The device is embedded into the fabric to maintain real-time sensor contact with the skin, enabling accurate physiological monitoring. The system features a docking connector that allows easy insertion, secure attachment, and electrical connectivity, including wireless charging and data transfer. Designed to be compatible with popular fitness devices, the glove, mitten, or sleeve also incorporates waterproofing and durability features, making it suitable for various environmental conditions. This invention enhances performance, safety, and convenience by providing seamless monitoring and feedback during dynamic physical activities.

### CROSS-REFERENCE TO RELATED APPLICATIONS

This application claims benefit to the earlier filing date and right of priority under 35 U.S.C. § 120 to patent application Ser. No. 17/756,337, the contents of which are herein incorporated by reference under 37 CFR 1.57 (b).

### TECHNICAL FIELD OF THE INVENTION

This invention relates generally to an active technology device enabled glove in the form of a strap, device, or apparatus for attaching an active technology device that is integrated into a glove for the purpose of permitting direct skin contact on the hand for optimized use of said active technology device while needing to wear/use gloves/hand-wear.

The invention relates generally to a sports glove with straps or mounts that secures wearable devices in the optimal position for viewing and interactive usage of a wearable device.

This invention relates generally to a wearable technology system. This invention relates more particularly to an apparatus/device for holding and integrating an active technology device (ATD) into sportswear.

This invention relates generally to apparatuses and devices for embedding and securing fitness and health tracking devices within wearable items. This method also can be used with various types of sports and fitness activities. This invention relates more particularly to a wearable system designed to enhance performance and safety by providing real-time monitoring and feedback.

### BACKGROUND

The present state of the art prior to this invention involves various attempts to integrate technology, such as sensors, fitness trackers, and communication devices, into wearable items like gloves and wristbands. These prior art solutions focus on different aspects of integrating technology into wearables, with some key trends and limitations:

Fitness Tracking Devices: Devices like smartwatches (e.g., Apple Watch, Fitbit) and fitness trackers have become popular for monitoring health metrics such as heart rate, steps, and sleep patterns. These devices are typically worn on the wrist and offer a range of sensors for tracking physiological data during physical activities. Limitation: Traditional wrist-based wearables can interfere with certain sports and activities, where wrist motion or contact might disrupt monitoring accuracy. They may also suffer from viewing angle issues during activities that require rapid movement.

Gloves with Integrated Sensors: Prior art, such as US20120144554A1, discloses gloves with embedded sensors designed for specific purposes like fitness tracking, rehabilitation, or gaming. These gloves may include sensors for monitoring heart rate, oxygen levels, motion, or pressure, with data stored locally or transferred to external devices. Limitation: These gloves often focus on static applications or specific activities, and their sensor placement may not provide continuous and accurate data during dynamic movements. The integration of technology is typically limited to specific sensors, and does not address broader usability challenges, such as real-time visibility of fitness data or seamless interaction during sports.

Wearable Communication Devices: Devices like the glove-type mobile phone terminal in JP2006165892A have attempted to integrate mobile communication features into gloves, with displays and input devices located on the hand. These wearables are designed for convenience, allowing users to interact with their devices without needing to remove them from a pocket or bag. Limitation: These communication-focused wearables are not designed for fitness tracking or health monitoring. Their integration of technology into gloves is limited to basic communication functions, and they lack the physiological monitoring capabilities required for fitness or sports applications.

Wrist-Based Wearables: Wrist-based devices, including smartwatches and fitness trackers, are the most common form of wearable technology for monitoring health metrics. They are widely adopted due to their ease of use and integration with smartphone apps for data tracking and analysis. Limitation: While effective for general fitness tracking, wrist-based wearables can be obstructive during certain activities, particularly those that involve frequent hand movements or contact with equipment. Additionally, their fixed position on the wrist can make it difficult to view data during dynamic activities, reducing their utility in high-performance sports.

### Overall Challenges in the Prior Art:

Limited Sensor Placement: Existing gloves and wearables often fail to maintain continuous and accurate sensor contact with the skin during vigorous activities, leading to interruptions in data monitoring.

Ergonomics and Viewing Angles: Devices integrated into gloves or worn on the wrist may not be positioned optimally for real-time visibility, forcing users to adjust their posture or stop their activity to check their data.

Connectivity and Power: Many existing devices either lack seamless connectivity features, such as wireless charging and data transfer, or they require manual intervention to sync data and manage power, which can be inconvenient during continuous activity.

Durability and Environmental Protection: While some devices may offer basic protection from the elements, few are designed for the rigors of intense physical activities in various weather conditions, limiting their reliability and usefulness in all environments.

In summary, prior art wearables provide basic functionality for fitness tracking or communication but often lack the comprehensive integration of real-time monitoring, ergonomic design, continuous connectivity, and environmental durability needed for high-performance sports and dynamic physical activities.

In light of the foregoing prior art, there is a need for a wearable system to better integrate real-time monitoring, ergonomic design, and seamless connectivity during dynamic sports and physical activities. Further, there is a need for a device that takes into consideration non-interference with activity and optimal viewing angle.

### BRIEF SUMMARY OF THE INVENTION

This invention is a specialized glove, mitten, or sleeve designed to securely hold an Active Technology Device (ATD), such as a smartwatch or fitness tracker, during sports activities. Positioned in the webbing between the thumb and forefinger, the ATD is placed at an optimal viewing angle to ensure continuous visibility and minimal interference with hand movements. The device is embedded into the fabric, ensuring that sensors maintain real-time contact with the skin for accurate physiological monitoring. The system includes a docking connector that allows for easy insertion, secure attachment, and seamless electrical connectivity, including wireless charging and data transfer. The design is compatible with popular fitness devices and incorporates waterproofing and durability features, making it suitable for various environmental conditions. This invention enhances performance and safety by providing uninterrupted monitoring and feedback during dynamic sports activities.

According to a first aspect of the invention, there is a glove comprising a dorsal hand side, a thumb sleeve, and a forefinger sleeve, with an empty space positioned over a webbing between a thumb and a forefinger, specifically configured to hold an active technology device (ATD) during a sport activity, wherein said ATD is placed at an optimal viewing angle, perpendicular to a line drawn from a first joint of said thumb to a first joint of said forefinger, with an area between said thumb and said forefinger being the least active part of a hand, reducing viewing errors and enhancing user performance and safety by maintaining an always-in-view perspective of said ATD during activity, wherein said ATD is embedded into said glove to allow sensors of said ATD to contact a skin of a user and enable real-time monitoring of physiological data, said configuration being distinct from prior glove-mounted sensors or phone terminals by ensuring uninterrupted sensor contact and visibility during dynamic sports activities.

According to a second aspect of the invention, there is a glove further comprising an ATD docking connector designed to securely hold and enable easy insertion and removal of said ATD, wherein said ATD docking connector allows for both secure attachment and electrical connectivity to said ATD, thereby supporting power supply and data exchange, which ensures continued functionality without requiring said user to handle said ATD separately during physical activities.

According to a third aspect of the invention, there is a glove wherein said ATD is selected from a group consisting of an Apple Watch, a Fitbit, a Garmin Smartwatch, and a Samsung Smartwatch, with said glove designed to accommodate devices specifically intended for fitness tracking and health monitoring, distinguishing said glove from gloves that incorporate communication devices such as mobile phones.

According to a fourth aspect of the invention, there is a glove wherein said ATD docking connector integrates wireless charging capabilities and is configured to interface with said ATD in a manner that allows said ATD to perform tracking and monitoring functions even during vigorous physical activity, differentiating from other gloves with simple electrical connections or sensor integration.

According to a fifth aspect of the invention, there is a mitten comprising a dorsal hand side, a thumb sleeve, and a forefinger sleeve, with an empty space positioned over a webbing between a thumb and a forefinger, specifically configured to hold an active technology device (ATD) during a sport activity, wherein said ATD is placed at an optimal viewing angle, perpendicular to a line drawn from a first joint of said thumb to a first joint of said forefinger, with an area between said thumb and said forefinger being the least active part of a hand, reducing viewing errors and enhancing user performance and safety by maintaining an always-in-view perspective of said ATD during activity, with real-time sensor contact maintained throughout use.

According to a sixth aspect of the invention, there is a mitten further comprising an ATD docking connector designed to securely hold and enable easy insertion and removal of said ATD, wherein said ATD docking connector provides electrical connectivity and is designed for seamless integration with charging and data transfer mechanisms of said ATD, improving user experience during extended wear, unlike prior art solutions that focus on integrating communication devices.

According to a seventh aspect of the invention, there is a mitten wherein said ATD is selected from a group consisting of an Apple Watch, a Fitbit, a Garmin Smartwatch, and a Samsung Smartwatch, ensuring compatibility with mainstream fitness and health tracking devices, distinguishing said mitten from mittens that integrate communication-specific devices such as mobile phones.

According to an eighth aspect of the invention, there is a mitten wherein said ATD docking connector integrates both wired and wireless data communication capabilities, allowing continuous data synchronization without the need for manual intervention during activities, overcoming the limitations of prior art that only provided basic connectivity for specific device functions.

According to a ninth aspect of the invention, there is a sleeve comprising a dorsal hand side, a thumb sleeve, and a forefinger sleeve, with an empty space positioned over a webbing between a thumb and a forefinger, specifically configured to hold an active technology device (ATD) during a sport activity, wherein said ATD is placed at an optimal viewing angle, perpendicular to a line drawn from a first joint of said thumb to a first joint of said forefinger, with an area between said thumb and said forefinger being the least active part of the hand, reducing viewing errors and enhancing user performance and safety by ensuring uninterrupted monitoring and feedback.

According to a tenth aspect of the invention, there is a sleeve further comprising an ATD docking connector designed to securely hold and enable easy insertion and removal of said ATD, with provisions for both physical and electrical integration that facilitate seamless power and data connectivity, unlike prior art gloves that incorporate communication or fitness-related devices in a less ergonomic configuration.

According to an eleventh aspect of the invention, there is a sleeve wherein said ATD is selected from a group consisting of an Apple Watch, a Fitbit, a Garmin Smartwatch, and a Samsung Smartwatch, providing a more specialized approach to health and fitness tracking compared to prior art sleeves that focus on communication device integration.

According to a twelfth aspect of the invention, there is a sleeve wherein said ATD docking connector includes waterproofing features to protect said ATD and ensure consistent performance in varying environmental conditions, a feature not emphasized in prior art devices that primarily focus on communication or sensor integration.

An advantage of the present invention is that it takes into consideration the usage of an active technology device in activities that require the wearing and use of gloves and how potential users interact with their wearable devices, not to simply see a device but the need to interact with it is as critical an aspect.

### Advantages of the Present Invention

Optimized Visibility and Ergonomics: The ATD is positioned in the least active part of the hand, allowing for optimal visibility and minimal interference with hand movements during sports activities.

Continuous Sensor Contact: The device is embedded into the glove, mitten, or sleeve, ensuring uninterrupted sensor contact with the skin, enabling real-time physiological monitoring.

Secure and Convenient Docking: The ATD docking connector securely holds the device while allowing for easy removal, and supports power supply and data exchange during activities.

Wireless Charging and Data Communication: Integration of wireless charging and seamless data communication eliminates the need for wired connections, enhancing convenience during physical activities.

Waterproof and Durable Design: Waterproofing and environmental protection features ensure reliable performance in various conditions, making the system suitable for intense sports and all weather.

Compatibility with Popular Devices: The design is compatible with major fitness tracking devices like the Apple Watch, Fitbit, Garmin, and Samsung Smartwatch, providing flexibility for users.

These features together enhance the user experience by providing continuous monitoring, convenience, and comfort during dynamic sports activities, setting this invention apart from prior art.

The invention will now be described, by way of example only, with reference to the accompanying drawings in which:

### BRIEF DESCRIPTION OF THE DRAWINGS

FIG. 1 is a top view of a glove showing an X over the area of the glove that covers the location of the muscles of thenar eminence in the area between the thumb and index finger of a hand according to the invention;

FIG. 2 is a top view of a glove showing a mounting and attachment loop sewn to glove according to the invention;

FIG. 3 is a top view of a glove showing a mounting and attachment loop sewn to glove holding an active technology device docking connector according to the invention;

FIG. 4 is a top view of a glove showing an active technology device connected to the glove configuration shown in FIG. 3 according to the invention;

FIG. 5 is a top view of a glove showing an access having an edge through said glove (hole through the glove) according to the invention;

FIG. 6 is a top view of a glove showing an access having an edge through said glove (hole through the glove) fitted with an active technology device docking connector according to the invention;

FIG. 7 is a top view of a glove showing use of an active technology device while wearing a bicycling glove according to the invention;

FIG. 8 is a top view of a glove showing use of an active technology device while wearing a bicycling glove according to the invention;

FIG. 9 is a perspective view from the top of a prototype active technology device docking connector as an Apple® watch holder according to the invention;

FIG. 10 is a perspective view from the bottom of a prototype active technology device docking connector as an Apple® watch holder with an Apple® watch device inserted therein according to the invention;

FIG. 11 is a top view of a mitten showing a mounting and attachment loop sewn to glove holding an active technology device docking connector according to the invention; and

FIG. 12 is a top view of a sleeve showing a mounting and attachment loop sewn to glove holding an active technology device docking connector according to the invention.

### DETAILED DESCRIPTION

The detailed embodiments of the present invention are disclosed herein. The disclosed embodiments are merely exemplary of the invention, which may be embodied in various forms. The details disclosed herein are not to be interpreted as limiting, but merely as the basis for the claims and as a basis for teaching one skilled in the art how to make and use the invention.

References in the specification to “one embodiment,” “an embodiment,” “an example embodiment,” etcetera, indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described.

Furthermore, it should be understood that spatial descriptions (e.g., “above,” “below,” “up,” “left,” “right,” “down,” “top,” “bottom,” “vertical,” “horizontal,” etc.) used herein are for purposes of illustration only, and that practical implementations of the structures described herein can be spatially arranged in any orientation or manner.

Throughout this specification, the word “comprise”, or variations thereof such as “comprises” or “comprising”, will be understood to imply the inclusion of a stated element, integer or step, or group of elements integers or steps, but not the exclusion of any other element, integer or step, or group of elements, integers or steps.

Throughout this specification, the word “active technology device”, or variations thereof such as “ATD”, will be understood to imply wearable devices and the various smart devices in use by people today with direct human interface technology to include, but not be limited to the Apple Watch, Samsung Galaxy Watch Active, Fossil the Sport Smartwatch, Fitbit devices, as well as other commercially available smart watches and smart devices.

Throughout this specification, the word “docking connector”, or variations thereof, will be understood to imply the inclusion of any active technology device attachment form, template, frame, outline, or sewn-in edge that enables an ATD to be inserted therein and positioned for optimal use orientation.

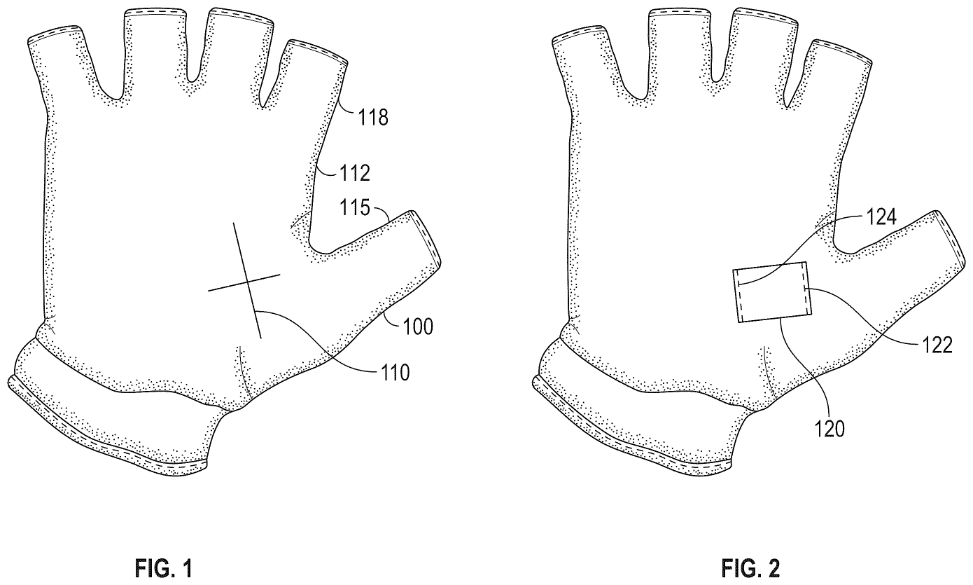

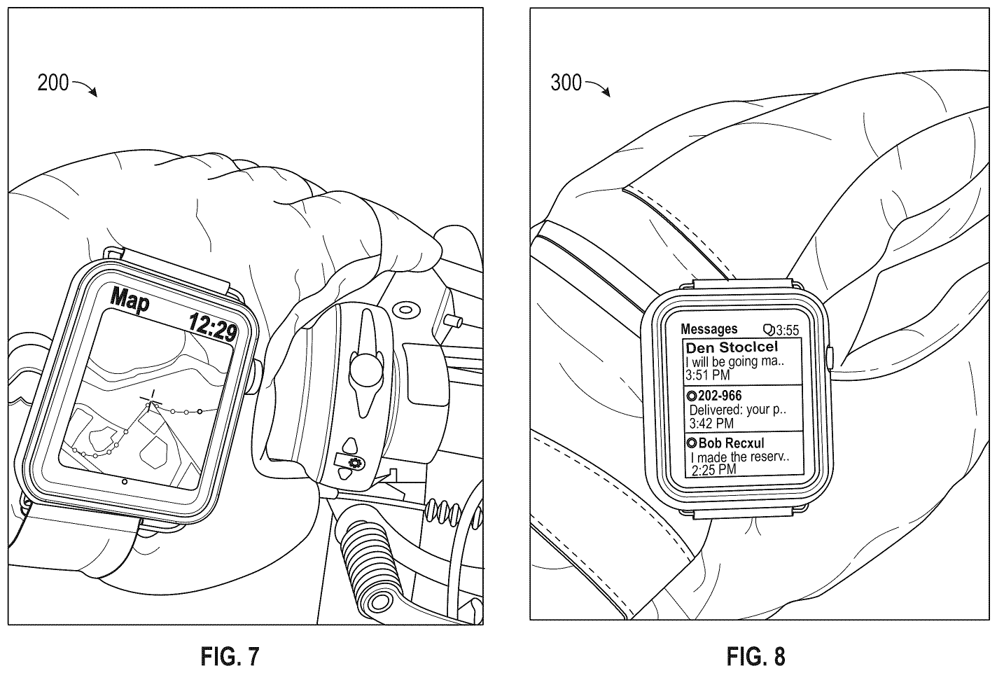

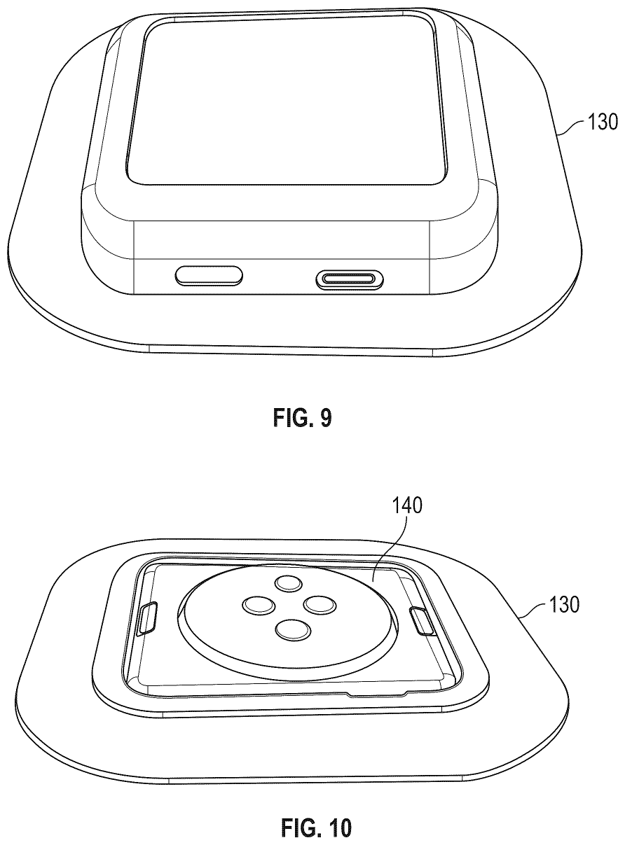

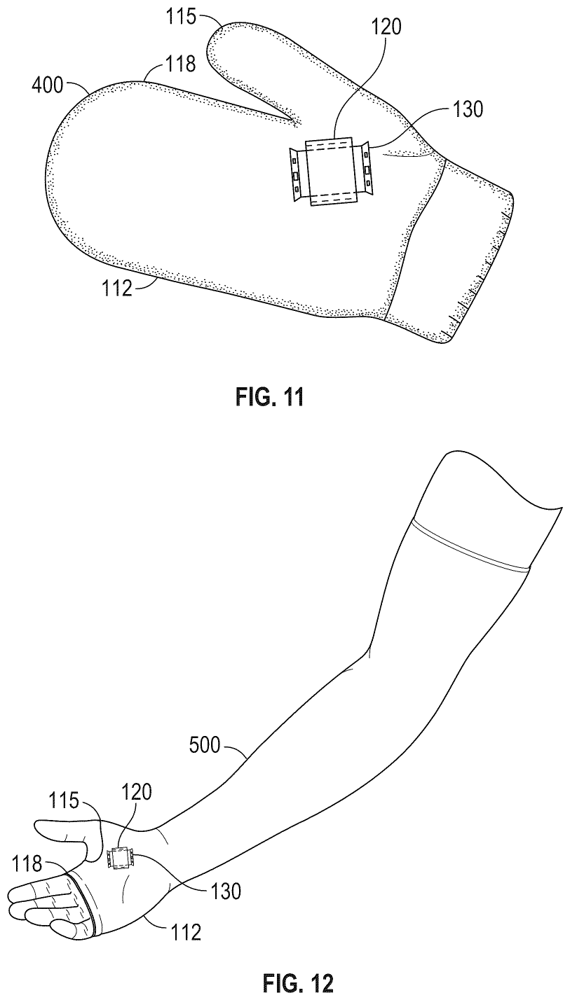

Index of Labelled Features in Figures. Features are listed in numeric order by Figure in numeric order. Referring to the Figures, there is shown in FIGS. 1 through 10 the following features:
Element 100 which is a glove.
Element 110 which is an empty space positioned over a webbing between a thumb and a forefinger.
Element 112 which is a dorsal hand side.
Element 115 which is a thumb sleeve.
Element 118 which is a forefinger sleeve.
Element 120 which is a strap or mount that secures wearable devices in the optimal position for viewing and use.
Element 122 and Element 124 are attachments at the ends of the strap 120 to fix said strap 120 to a glove 100.
Element 130 which is an active technology device docking connector.
Element 140 which is an active technology device.
Element 150 which is an access through the glove.
Element 155 which is a mounting or attachment edge for an access through the glove 150.
Element 160 which is an insertable active technology device docking connector.
Element 200 which is a bicycling glove with active technology device attached according to the present invention.
Element 300 which is a skiing glove with active technology device attached according to the present invention.
Element 400 which is a mitten.
Element 500 which is a sleeve.

When you typically wear a wearable device like an Apple Watch you fasten it to your wrist. Then when doing an activity like skiing you put on a thermal shirt over it then possibly a fleece then a Ski Jacket, and gloves. When you attempt to access the device, it is either very difficult or not reasonably possible to quickly do without removing clothing. The active glove mounting of the wearable according to the present invention eliminates this problem.

The first problem was to define the exact placement where a wearable device is most visible, protected from movement and ultimately most accessible. That was outside the clothing and on the glove and aligned on the back of the glove between the first joint of the thumb and index finger. This specific placement will not interfere with the user's activities and provides the needed accessibility.

The second problem was to design an attachment device that would accommodate a variety of wearables and not require specific gloves for specific devices. A glove with a specific holder is still a desirable outcome however not limited to that requirement. The attachment device as illustrated allows for most devices to be mounted in the specific least active area on the glove and provides acceptable shock absorption from the user's activities.

Although the strap provides the flexibility of attaching a wide variety of devices it is still preferred to have gloves with a insert that provides both protection and when imbedded into the glove or mitten and will also contact the users skin.

The specific placement of the wearable device is the most critical aspect of the invention. It is also described as area between the thumb and index finger (digitus secundus manus, pointer finger, or forefinger), is called the thenar space. This precise placement provides a secure and unobstructed view of the device and is positioned in a protected area from most active movements. This area is illustrated in FIG. 1 as element 110, the location of the area of a glove that is over the muscles of thenar eminence of a hand when using said glove. Other locations on the outer side of the glove are not ideal and actually interfere with many intended activities.

The present invention is directed to a glove with straps or mounts that secures wearable ATD devices in the optimal position for viewing and use while wearing gloves and actively involved in an activity.

By imbedding the device through the glove so contact with the skin is allowed the sensors for pulse, heart rate and oxygen are able to function/operate properly. The present invention is not limited to these sensors however and can accommodate many other options commonly implemented in wearable devices both currently in production or being developed. The Figures show the use of an Apple Watch holder but the present invention is not limited to Apple Devices. Holders (docking connector 130) can be provided for all available wearable devices.

Although it is described as a glove it is not limited to a specific type of glove and or mitten. Full gloves, fingerless gloves, half fingers, sleeves with thumb loops as well as other types of gloves that cover the specific placement area of the back of the hand are covered under this description.

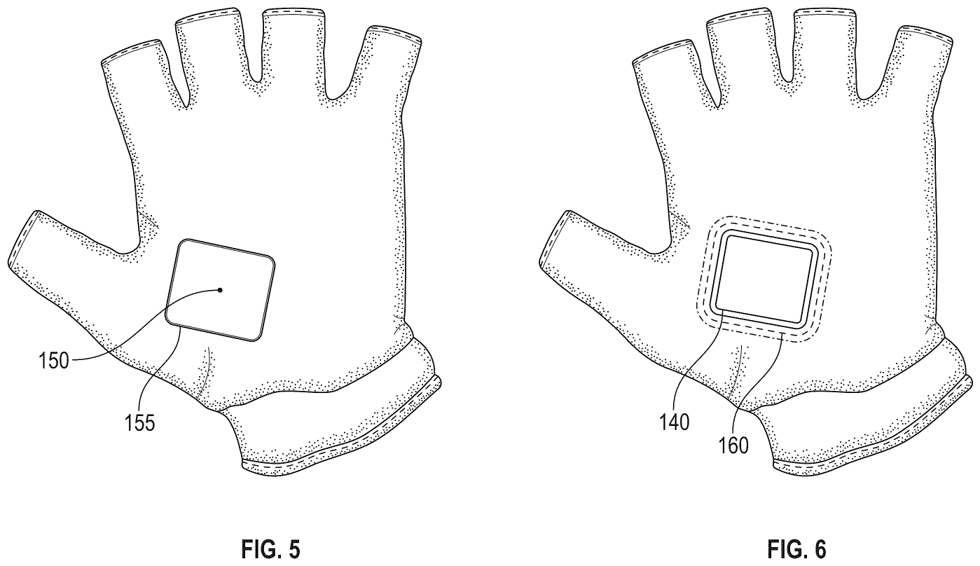

The imbedded wearable holder would be best made of clear TPU but is not limited to this material. Any suitable material commonly used for wearable device protection and or used in the production of athletic equipment or sports gloves/mittens can be used. The holders could be stitched, glued, welded or fused to the underlying material, but is not limited to these methods. Methods commonly used in production of sports equipment or athletic gloves could otherwise be used to attached to the glove/mitten. FIGS. 5 and 6 illustrate the concept of the imbedded device holder (imbedded docking connector 160).

The attachment straps would be made of two components one a flexible webbing material but not limited to this material. Any material approximately 48 mm in width and 40 mm that could be attached to the glove would be possible. This component is attached to the back of the Glove or Mitten in the precise location as previously described. Stitched, glued, welded/fused to the underlying material on each side of the 48 mm with to create a open loop for the second component to be passed through.

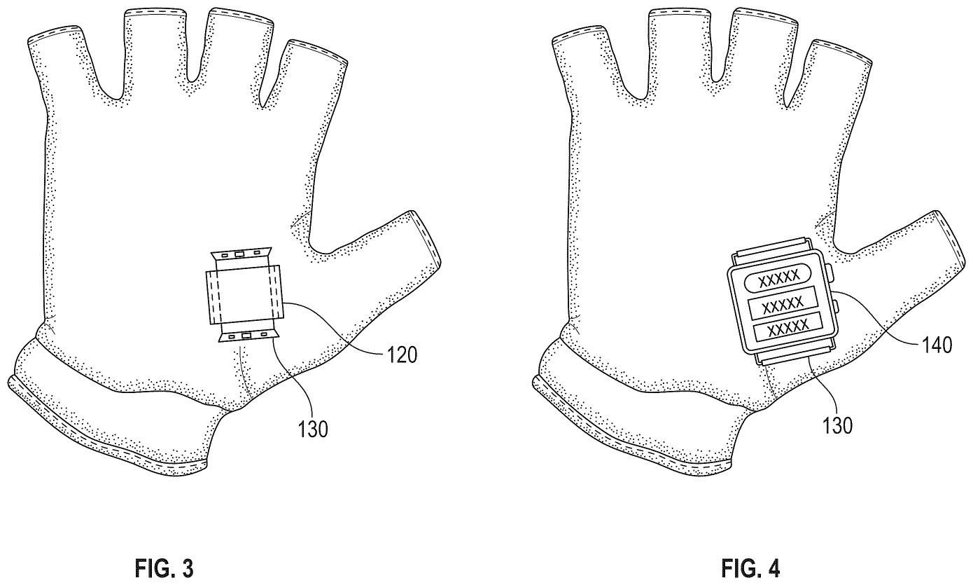

The second component of the strap is made of industrial elastic but not limited to this material, approximately 20 mm wide and 60 mm in length. The second component is sewn at each end to create an open loop that can have an attachment PIN for devices like the Garmin or Apple Watch compatible connector installed. These connectors are not limited to these but can as well accommodate others that are commonly used in wearable devices. FIG. 3 illustrates the active strap installed with the active loop.

The specific placement of the wearable device is the most critical aspect of the invention. It is also described as area between the thumb and index finger (digitus secundus manus, pointer finger, or forefinger), is called the thenar space. This precise placement provides a secure and unobstructed view of the device and is positioned in a protected area from most active movements. The concept of reducing viewing errors by placing an ATD in a non-moving, easily viewable location on a glove, mitten, or sleeve is considered obvious to a person skilled in the art. This is because:

Stable Positioning: By securing the ATD in a fixed position, the user can quickly and reliably glance at the device without the need for adjustments. This stability is crucial during dynamic sports activities where movements are frequent and rapid.

Ergonomic Design: The placement of the ATD in an accessible location aligns with ergonomic principles, ensuring that the device is within the user's natural line of sight. This minimizes the effort required to view the device, thereby reducing the chance of errors due to misalignment or movement.

Enhanced Usability: A non-moving location ensures that the device remains consistently visible and accessible, enhancing the overall usability and effectiveness of the ATD during sports activities.

The two main reasons for the location of the ATD is non-interference with activity and optimal viewing angle.

Non-Interference with Activity: The specific location of the ATD does not interfere with sports activities or equipment.

Optimal Viewing Angle: The ATD is mounted at an optimal angle for viewing, perpendicular to a line drawn from the first joint of the thumb to the first joint of the index finger. This placement, above the webbing between the thumb and forefinger, is the best option for ATD usage.

By integrating the ATD into the glove, mitten, or sleeve and allowing contact with the user's skin, all functionalities of the ATD are accessible.

The imbedded wearable holder would be best made of clear TPU but is not limited to this material. Any suitable material commonly used for wearable device protection and or used in the production of athletic equipment or sports gloves/mittens can be used. The holders could be stitched, glued, welded or fused to the underlying material, but is not limited to these methods. 0052 Methods commonly used in production of sports equipment or athletic gloves could otherwise be used to attached to the glove/mitten.

### How to Make the Invention:

### Materials Selection:

Fabric and Components: Choose durable, flexible, and breathable materials for the glove, mitten, or sleeve, such as neoprene, spandex, or a moisture-wicking synthetic fabric. Ensure the materials are skin-friendly and capable of withstanding repeated wear and washing.

Docking Connector: Design a docking connector made of sturdy yet flexible plastic or silicone to securely hold the Active Technology Device (ATD) in place. The connector should have internal electrical contacts for power and data exchange, along with mechanisms for wireless charging if needed.

Waterproof Coating: Apply a waterproof coating to the fabric, particularly around the docking area, to protect the embedded device from sweat, rain, or other moisture. Ensure that the coating does not affect the fabric's flexibility or breathability.

Wiring and Electronics: Integrate thin, flexible wiring within the glove, mitten, or sleeve to connect the docking connector to a power source or data transfer module. Use waterproof, insulated wires to prevent damage.

### Design and Layout:

ATD Positioning: Create a designated space for the ATD in the webbing between the thumb and forefinger. This space should have enough room to accommodate different ATD models while ensuring that the device remains securely in place and easily visible.

Sensor Integration: If the ATD uses sensors (e.g., heart rate monitors), ensure that the docking connector positions the sensors in direct contact with the skin. The fabric around this area should be thin enough to allow the sensors to function correctly while still providing durability and comfort.

Attachment Method: Design the docking connector with a mechanism that allows for easy insertion and removal of the ATD. This can include snap-in designs, magnetic attachments, or a friction fit. Ensure that the attachment method is strong enough to keep the device secure during physical activity.

Power and Data Connection: Integrate a small battery and power management system within the glove, mitten, or sleeve if the device requires charging during use. The system should also support data transfer through wired or wireless connections (e.g., Bluetooth or NFC) without requiring the user to remove the device.

### Manufacturing Process:

Cut and Assemble Fabric: Cut the fabric to the desired shape for the glove, mitten, or sleeve. Assemble the pieces using stitching, bonding, or seamless fusing technology to create a comfortable and ergonomic fit.

Integrate Docking Connector: Attach the docking connector to the fabric using reinforced stitching, adhesive bonding, or integration into the fabric mold. Ensure the connector is securely anchored and aligned for correct device placement.

Embed Electronics: Place the wiring, battery, and other electronic components within the fabric layers, ensuring they are securely insulated and protected from wear and tear. Test all connections to ensure proper functionality before final assembly.

Apply Waterproof Coating: Apply the waterproof coating to all external surfaces, particularly around the docking connector and any electronic components, to ensure long-term durability in various environmental conditions.

Quality Testing: Test the final product for comfort, durability, and functionality, including the secure attachment of the ATD, sensor accuracy, and data connectivity. Conduct environmental tests to confirm waterproofing and resistance to wear.

### How to Use the Invention:

### Inserting the ATD:

Place your Active Technology Device (e.g., smartwatch, fitness tracker) into the designated docking connector on the glove, mitten, or sleeve. Ensure that the device snaps into place securely, with its sensors in contact with the skin.

If the docking connector supports wireless charging, ensure that the device is aligned correctly for charging while in use.

### Wearing the Glove, Mitten, or Sleeve:

Slide your hand into the glove, mitten, or sleeve as you would with a regular piece of sportswear. Adjust the fit as necessary to ensure comfort and proper placement of the ATD in the webbing between your thumb and forefinger.

Ensure that the fabric is snug but not too tight, allowing for freedom of movement while keeping the device securely in place.

### Using During Activities:

Once the ATD is in place, engage in your desired sports or physical activity. The ergonomic design ensures that the device remains in view at all times, allowing you to check your fitness data, such as heart rate, step count, or notifications, without interrupting your movements.

The device's sensors will continuously monitor your physiological data (e.g., heart rate, oxygen levels) as you exercise. Real-time data will be collected and displayed on the ATD screen, which is visible in its optimal placement.

### Data Transfer and Charging:

After completing your activity, if the ATD has wireless data transfer capabilities, your workout data will automatically sync to your connected devices (e.g., smartphone or computer) via Bluetooth, Wi-Fi, or NFC.

If your device needs charging, the glove, mitten, or sleeve's integrated charging system will power the device wirelessly or through a wired connection, depending on the model. You can charge the device without removing it from the docking connector.

### Removal and Maintenance:

After use, remove the glove, mitten, or sleeve, and if necessary, detach the ATD by releasing the docking connector. The easy-release mechanism allows for quick removal without damage to the device or the fabric.

Clean the glove, mitten, or sleeve according to the manufacturer's instructions, typically by hand-washing or using a gentle machine cycle. Ensure that the waterproof coating and electronic components remain intact during cleaning.

### Reusability and Storage:

Store the glove, mitten, or sleeve in a dry place, ensuring the ATD is either detached or securely docked if still in use. The durable materials and waterproof coating ensure that the system is ready for repeated use under various conditions.

This invention enhances the experience of athletes and active individuals by providing an ergonomic, functional, and durable solution for integrating fitness and health monitoring devices into sportswear. The system's design allows users to track their performance effortlessly, stay connected, and remain focused on their activities.

According to a preferred embodiment of the invention, there is a glove comprising a dorsal hand side, a thumb sleeve, and a forefinger sleeve, with an empty space positioned over a webbing between a thumb and a forefinger, specifically configured to hold an active technology device (ATD) during a sport activity, wherein said ATD is placed at an optimal viewing angle, perpendicular to a line drawn from a first joint of said thumb to a first joint of said forefinger, with an area between said thumb and said forefinger being the least active part of a hand, reducing viewing errors and enhancing user performance and safety by maintaining an always-in-view perspective of said ATD during activity, wherein said ATD is embedded into said glove to allow sensors of said ATD to contact a skin of a user and enable real-time monitoring of physiological data, said configuration being distinct from prior glove-mounted sensors or phone terminals by ensuring uninterrupted sensor contact and visibility during dynamic sports activities.

According to an alternate embodiment of the invention, there is a glove further comprising an ATD docking connector designed to securely hold and enable easy insertion and removal of said ATD, wherein said ATD docking connector allows for both secure attachment and electrical connectivity to said ATD, thereby supporting power supply and data exchange, which ensures continued functionality without requiring said user to handle said ATD separately during physical activities.

According to an alternate embodiment of the invention, there is a glove wherein said ATD is selected from a group consisting of an Apple Watch, a Fitbit, a Garmin Smartwatch, and a Samsung Smartwatch, with said glove designed to accommodate devices specifically intended for fitness tracking and health monitoring, distinguishing said glove from gloves that incorporate communication devices such as mobile phones.

According to an alternate embodiment of the invention, there is a glove wherein said ATD docking connector integrates wireless charging capabilities and is configured to interface with said ATD in a manner that allows said ATD to perform tracking and monitoring functions even during vigorous physical activity, differentiating from other gloves with simple electrical connections or sensor integration.

According to a preferred embodiment of the invention, there is a mitten comprising a dorsal hand side, a thumb sleeve, and a forefinger sleeve, with an empty space positioned over a webbing between a thumb and a forefinger, specifically configured to hold an active technology device (ATD) during a sport activity, wherein said ATD is placed at an optimal viewing angle, perpendicular to a line drawn from a first joint of said thumb to a first joint of said forefinger, with an area between said thumb and said forefinger being the least active part of a hand, reducing viewing errors and enhancing user performance and safety by maintaining an always-in-view perspective of said ATD during activity, with real-time sensor contact maintained throughout use.

According to an alternate embodiment of the invention, there is a mitten further comprising an ATD docking connector designed to securely hold and enable easy insertion and removal of said ATD, wherein said ATD docking connector provides electrical connectivity and is designed for seamless integration with charging and data transfer mechanisms of said ATD, improving user experience during extended wear, unlike prior art solutions that focus on integrating communication devices.

According to an alternate embodiment of the invention, there is a mitten wherein said ATD is selected from a group consisting of an Apple Watch, a Fitbit, a Garmin Smartwatch, and a Samsung Smartwatch, ensuring compatibility with mainstream fitness and health tracking devices, distinguishing said mitten from mittens that integrate communication-specific devices such as mobile phones.

According to an alternate embodiment of the invention, there is a mitten wherein said ATD docking connector integrates both wired and wireless data communication capabilities, allowing continuous data synchronization without the need for manual intervention during activities, overcoming the limitations of prior art that only provided basic connectivity for specific device functions.

According to a preferred embodiment of the invention, there is a sleeve comprising a dorsal hand side, a thumb sleeve, and a forefinger sleeve, with an empty space positioned over a webbing between a thumb and a forefinger, specifically configured to hold an active technology device (ATD) during a sport activity, wherein said ATD is placed at an optimal viewing angle, perpendicular to a line drawn from a first joint of said thumb to a first joint of said forefinger, with an area between said thumb and said forefinger being the least active part of the hand, reducing viewing errors and enhancing user performance and safety by ensuring uninterrupted monitoring and feedback.

According to an alternate embodiment of the invention, there is a sleeve further comprising an ATD docking connector designed to securely hold and enable easy insertion and removal of said ATD, with provisions for both physical and electrical integration that facilitate seamless power and data connectivity, unlike prior art gloves that incorporate communication or fitness-related devices in a less ergonomic configuration.

According to an alternate embodiment of the invention, there is a sleeve wherein said ATD is selected from a group consisting of an Apple Watch, a Fitbit, a Garmin Smartwatch, and a Samsung Smartwatch, providing a more specialized approach to health and fitness tracking compared to prior art sleeves that focus on communication device integration.

According to an alternate embodiment of the invention, there is a sleeve wherein said ATD docking connector includes waterproofing features to protect said ATD and ensure consistent performance in varying environmental conditions, a feature not emphasized in prior art devices that primarily focus on communication or sensor integration.

### Key Differentiators:

Specific Configuration for Sports: Unlike prior art that integrates general sensors or communication devices into gloves, the claims focus on a specific configuration that maintains visibility and functionality of health and fitness tracking devices during sports activities. Real-Time Sensor Contact: Emphasis on embedding the ATD into the fabric to ensure continuous skin contact and sensor functionality, which is not highlighted in prior art such as US20120144554A1.

Seamless Data Connectivity: The claims introduce both wired and wireless connectivity for uninterrupted data exchange, setting them apart from the mobile phone integration disclosed in JP2006165892A.

Ergonomic Design for Dynamic Use: The positioning of the ATD to optimize viewing angles and minimize interference during activity differentiates the invention from gloves that focus on static uses like communication.

These revisions aim to establish clear distinctions from the prior art while reinforcing the uniqueness of the invention in the context of sports and fitness activities.

Advantages of the present invention include:

Optimized Visibility and Ergonomics: Description: The Active Technology Device (ATD) is strategically positioned in the webbing between the thumb and forefinger, which is the least active part of the hand. This positioning ensures that the ATD is always visible to the user without requiring them to adjust their hand position. Advantage: This design minimizes interference with hand movements, making the system ideal for dynamic sports activities where freedom of motion is critical. The user can easily glance at their ATD for real-time data without sacrificing performance or safety.

Continuous Sensor Contact: Description: By embedding the ATD into the glove, mitten, or sleeve, the system ensures that the device's sensors maintain continuous contact with the skin. This allows for uninterrupted monitoring of physiological metrics such as heart rate, oxygen levels, and other health data. Advantage: Real-time sensor contact is essential for accurate data tracking, especially during vigorous activities. Unlike prior art, which may lose sensor contact during motion, this system guarantees consistent monitoring, enhancing the reliability of the health data collected.

Secure and Convenient Docking: Description: The ATD docking connector is designed to securely hold the device in place while allowing for easy insertion and removal. Additionally, the connector provides electrical connectivity, enabling power supply and data exchange during activities. Advantage: Users can securely attach their ATD and trust that it will remain in place during even the most strenuous activities. The ease of removal ensures convenience when the device needs to be taken off, while the electrical connectivity allows for continuous functionality, such as charging and data synchronization, without the need to handle the device separately.

Wireless Charging and Data Communication: Description: The system integrates wireless charging and data communication capabilities into the docking connector, allowing the ATD to be charged and synchronized wirelessly during use. Advantage: This feature eliminates the hassle of dealing with wired connections during physical activities, enabling the user to focus on their performance without worrying about their device running out of battery or needing to manually sync data. Wireless functionality enhances the user experience by adding convenience and reducing interruptions.

Waterproof and Durable Design: Description: The system includes waterproofing and environmental protection features that safeguard the ATD and ensure reliable performance in various conditions, including exposure to water, sweat, and extreme weather. Advantage: Durability is crucial for active users who engage in outdoor sports or activities in diverse environments. The waterproof design ensures that the device continues to function correctly even in rain or during intense sweat, making the system robust and dependable for all conditions.

Compatibility with Popular Devices: Description: The design is compatible with major fitness and health tracking devices, such as the Apple Watch, Fitbit, Garmin Smartwatch, and Samsung Smartwatch. Advantage: This compatibility makes the system highly versatile and user-friendly, allowing users to integrate their existing devices without needing to purchase new equipment. The system's ability to work with popular fitness trackers makes it accessible to a wide range of users, adding to its overall appeal.

Overall Impact: The combination of these features delivers a product that enhances the experience of users during sports and physical activities. By ensuring that the ATD remains visible, functional, and securely in place while providing uninterrupted sensor contact and wireless connectivity, the system allows users to focus on their performance while benefiting from real-time data tracking. The durable and waterproof design further ensures that the system can be used in various environments, making it a reliable companion for athletes and active individuals.

This set of advantages positions the invention as a superior solution for integrating fitness tracking and health monitoring devices into sportswear, overcoming the limitations of prior art and offering enhanced usability and convenience.

The invention has been described by way of examples only. Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the claims.

Although the invention has been explained in relation to various embodiments, it is to be understood that many other possible modifications and variations can be made without departing from the spirit and scope of the invention.

1. A glove comprising a dorsal hand side, a thumb sleeve, and a forefinger sleeve, with an empty space positioned over a webbing between a thumb and a forefinger, specifically configured to hold an active technology device (ATD) during a sport activity, wherein said ATD is placed at an optimal viewing angle, perpendicular to a line drawn from a first joint of said thumb to a first joint of said forefinger, with an area between said thumb and said forefinger being the least active part of a hand, reducing viewing errors and enhancing user performance and safety by maintaining an always-in-view perspective of said ATD during activity, wherein said ATD is embedded into said glove to allow sensors of said ATD to contact a skin of a user and enable real-time monitoring of physiological data, said configuration being distinct from prior glove-mounted sensors or phone terminals by ensuring uninterrupted sensor contact and visibility during dynamic sports activities.

2. The glove of claim 1, further comprising an ATD docking connector designed to securely hold and enable easy insertion and removal of said ATD, wherein said ATD docking connector allows for both secure attachment and electrical connectivity to said ATD, thereby supporting power supply and data exchange, which ensures continued functionality without requiring said user to handle said ATD separately during physical activities.

3. The glove of claim 1, wherein said ATD is selected from a group consisting of an Apple Watch, a Fitbit, a Garmin Smartwatch, and a Samsung Smartwatch, with said glove designed to accommodate devices specifically intended for fitness tracking and health monitoring, distinguishing said glove from gloves that incorporate communication devices such as mobile phones.

4. The glove of claim 2, wherein said ATD docking connector integrates wireless charging capabilities and is configured to interface with said ATD in a manner that allows said ATD to perform tracking and monitoring functions even during vigorous physical activity, differentiating from other gloves with simple electrical connections or sensor integration.

5. A mitten comprising a dorsal hand side, a thumb sleeve, and a forefinger sleeve, with an empty space positioned over a webbing between a thumb and a forefinger, specifically configured to hold an active technology device (ATD) during a sport activity, wherein said ATD is placed at an optimal viewing angle, perpendicular to a line drawn from a first joint of said thumb to a first joint of said forefinger, with an area between said thumb and said forefinger being the least active part of a hand, reducing viewing errors and enhancing user performance and safety by maintaining an always-in-view perspective of said ATD during activity, with real-time sensor contact maintained throughout use.

6. The mitten of claim 5, further comprising an ATD docking connector designed to securely hold and enable easy insertion and removal of said ATD, wherein said ATD docking connector provides electrical connectivity and is designed for seamless integration with charging and data transfer mechanisms of said ATD, improving user experience during extended wear, unlike prior art solutions that focus on integrating communication devices.

7. The mitten of claim 5, wherein said ATD is selected from a group consisting of an Apple Watch, a Fitbit, a Garmin Smartwatch, and a Samsung Smartwatch, ensuring compatibility with mainstream fitness and health tracking devices, distinguishing said mitten from mittens that integrate communication-specific devices such as mobile phones.

8. The mitten of claim 6, wherein said ATD docking connector integrates both wired and wireless data communication capabilities, allowing continuous data synchronization without the need for manual intervention during activities, overcoming the limitations of prior art that only provided basic connectivity for specific device functions.

9. A sleeve comprising a dorsal hand side, a thumb sleeve, and a forefinger sleeve, with an empty space positioned over a webbing between a thumb and a forefinger, specifically configured to hold an active technology device (ATD) during a sport activity, wherein said ATD is placed at an optimal viewing angle, perpendicular to a line drawn from a first joint of said thumb to a first joint of said forefinger, with an area between said thumb and said forefinger being the least active part of the hand, reducing viewing errors and enhancing user performance and safety by ensuring uninterrupted monitoring and feedback.

10. The sleeve of claim 9, further comprising an ATD docking connector designed to securely hold and enable easy insertion and removal of said ATD, with provisions for both physical and electrical integration that facilitate seamless power and data connectivity, unlike prior art gloves that incorporate communication or fitness-related devices in a less ergonomic configuration.

11. The sleeve of claim 9, wherein said ATD is selected from a group consisting of an Apple Watch, a Fitbit, a Garmin Smartwatch, and a Samsung Smartwatch, providing a more specialized approach to health and fitness tracking compared to prior art sleeves that focus on communication device integration.

12. The sleeve of claim 10, wherein said ATD docking connector includes waterproofing features to protect said ATD and ensure consistent performance in varying environmental conditions, a feature not emphasized in prior art devices that primarily focus on communication or sensor integration.
